(19) United States
(12) Plant Patent Application Publication
(10) Pub. No.: US 2005/0257307 P1
(43) Pub. Date: Nov. 17, 2005

(54) New guinea impatiens plant named 'Danhardalil'

(50) IMPATIENS HAWKERII; NEW GUINEA IMPATIENS

(75) Inventor: Gabriel Danziger, Moshav Nir-Zvi (IL)

Correspondence Address: FOLEY AND LARDNER;SUITE 500, 3000 K STREET NW, WASHINGTON, DC, 20007, US

(73) Assignee: Danziger "Dan" Flower Farm

(21) Appl. No.: 10/846,635

(22) Filed: May 17, 2004

(51) Int. Cl. A01H 5/00

(52) U.S. Cl. PLT/318

(57) ABSTRACT

A new and distinct Impatiens plant named ‘Danhardalil’ characterized by having large, round flowers; dark lilac flower color; green leaves, mature leaf upper surface RHS 147 A; and compact and round plant habit.

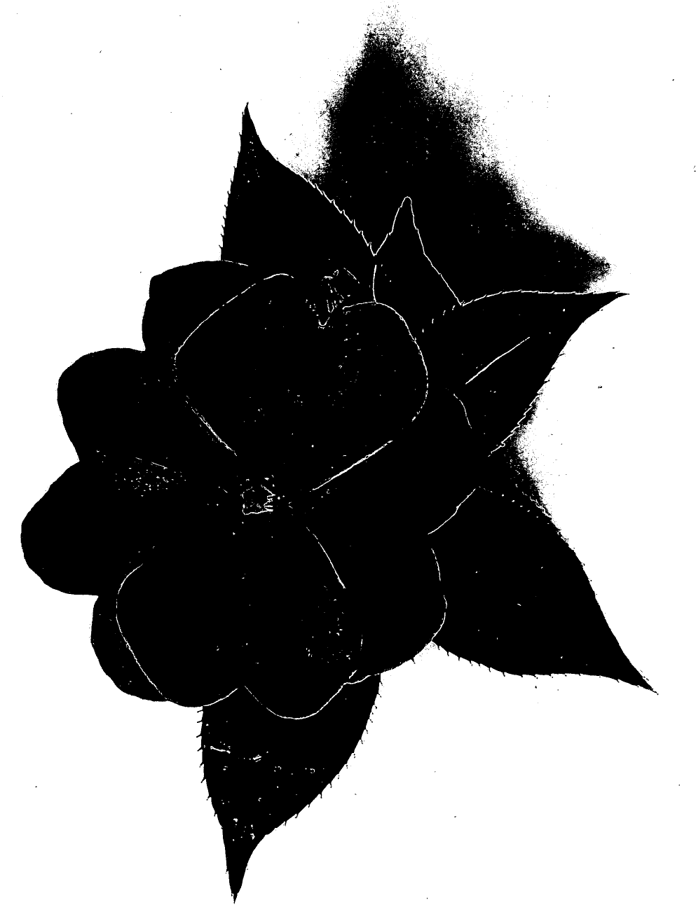

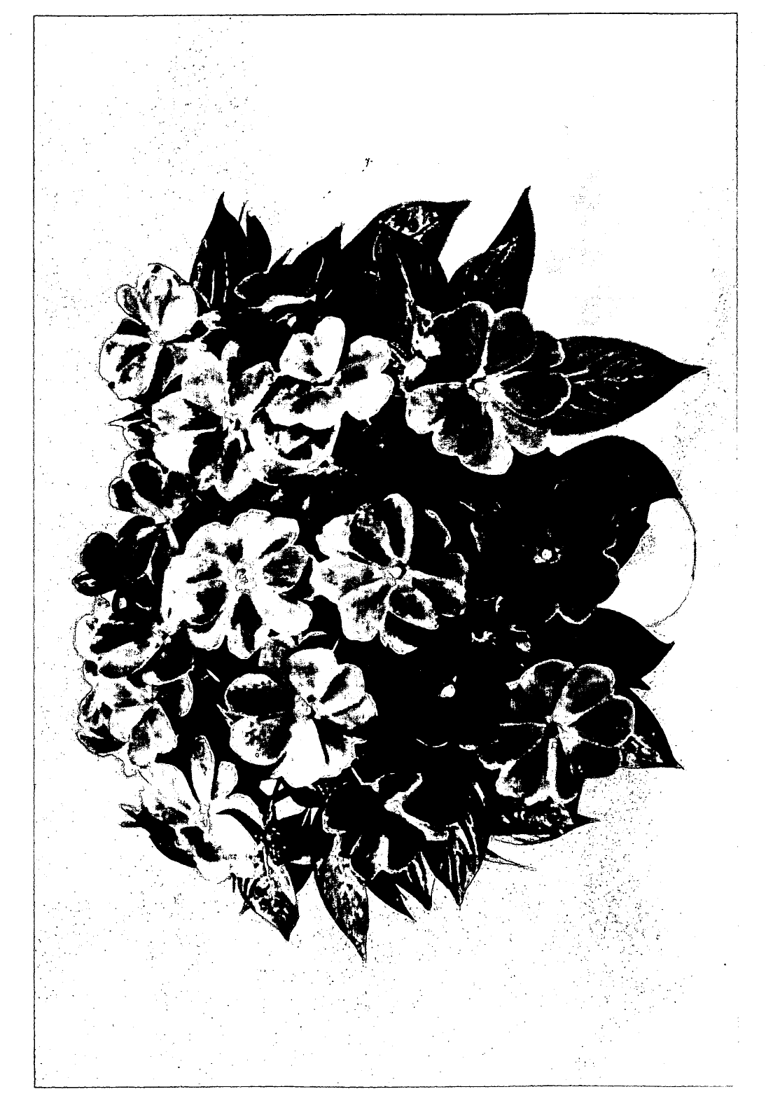

### GENUS AND SPECIES OF THE PLANT CLAIMED

Impatiens

### VARIETY DENOMINATION

‘Danhardalil’

### BACKGROUND OF THE INVENTION

The present invention relates to a new and distinct cultivar of New Guinea Impatiens plant, botanically known as Impatiens, hereinafter referred to by the cultivar name “Danhardalil”.

The new cultivar originated from a cross made in a controlled breeding program in Moshav Mishmar Hashiva, Israel. The female parent is a proprietary cultivar designated H-243 (unpatented). The male parent is a proprietary cultivar designated H-202 (unpatented). ‘Danhardalil’ was discovered and selected by the inventor, Gabriel Danziger, as a flowering plant within the progeny of the stated cross in a controlled environment in. Moshav Mishmar Hashiva, Israel.

Asexual reproduction of the new cultivar was performed by leaf cuttings. The cuttings are apical cuttings; no more than two expanded leaves and three to four immature leaves evident. Asexual reproduction of ‘Danhardalil’ was first performed in December, 2001, in Moshav Mishmar Hashiva, Israel, and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Danhardalil’ which in combination distinguish this Impatiens as a new and distinct cultivar:

1. Large, round flower;

2. dark lilac flower color;

3. green leaves, mature leaf upper surface RHS 147 A; and

4. compact and round plant habit.

‘Danhardalil’ has not been observed under all possible environmental conditions. The phenotype of the new cultivar may vary significantly with variations in environment such as temperature, light intensity, and daylength without any change in the genotype of the plant. The following observations, measurements and values describe the new cultivar as grown in Moshav Mishmar Hashiva, Israel under conditions which closely approximate those generally used in commercial practice.

Of the many commercial cultivars known to the inventor, the most similar in comparison to ‘Danhardalil’ is cultivar ‘Harmony Dark Lavender’ (unpatented). ‘Danhardalil’ has a larger flower than ‘Harmony Dark Lavender’, and the flower color of ‘Danhardalil’ is darker than that of ‘Harmony Dark Lavender’.

### BRIEF DESCRIPTION OF THE DRAWING

The accompanying photographic drawing shows colors as true as is possible with reproductions of this nature. The first photograph shows a ‘Danhardalil’ plant in its entirety. The second photograph shows a close-up of a ‘Danhardalil’ flower and leaves.

### DETAILED BOTANICAL DESCRIPTION

The following observations, measurements and values describe the new cultivar as grown in Moshav Mishmar Hashiva, Israel under conditions which closely approximate those generally used in commercial practice: day temperature 20 C to 24 C, night temperature 18 C to 20 C, fertilized up to a level of 150-200 ppm N. 80 ppm P, 150 ppm K. Color references are made to The Royal Horticultural Society Colour Chart (RHS), except where general colors of ordinary significance are used. Color values were taken under daylight conditions at approximately 1:00 pm in a greenhouse.

### PLANT

General appearance and form:
   Height.—15 cm.
   Width.—30 cm.
   Habit.—Round and compact, very good branching.
   Flowering response.—Day natural.
   Flowering season.—All year round.
   Winter hardiness.—Medium.
   Lastingness of the individual bloom.—Floral life ranged 5-10 days after anthesis.
   Rooting.—Time to initiate roots 7-10 days in summer time (25 C), 10-14 days in winter (20 C); no hormone required for rooting.
   Fragrance.—None.

### FOLIAGE

Overall shape of leaf: Lanceolate ovate.
Base: Acuminate.
Tip: Attenuate.
Margin: Ciliate.
Texture: Smooth.
Color of upper surface:
   Mature leaf.—RHS-147 A.
   Immature leaf.—RHS-137 A.
Color of lower surface:
   Mature leaf.—Green RHS 137 C.
   Immature leaf.—Green RHS 138 B.
Venation color:
   Upper surface.—Green RHS 138B.
   Lower surface.—Green RHS 138B.
Size:
   Length.—10-13 cm.
   Width.—4 cm.

### INFLORESCENCE

Corolla:
   Form.—Single.
   Shape.—Round.
   Number of blooms.—20-25 flowers per mature plant [4 months old].
   Size.—8-8.5 cm Petal Number: 5.
   Petal shape.—Spatulate.
   Petal color.—Upper surface: Purple RHS N 78B Lower surface: Purple RHS N 78C.
Stem:
   Average length.—13 cm.
   Average diameter.—0.5 cm.
   Color.—Green RHS 144 B.
   Internode length.—4 cm.
Spur:
   Color.—Greyed red RHS 182C.
Bud:
   Response.—10-14 days.
   Color.—Purple RHS N 78B.
   Size before opening.—2.5 cm.
   Aspect.—Oval.
   Pedicel length.—5.5 cm.
Pedicel color: Yellow green group RHS 144B.
Reproductive organs:
Stamen: 1; white in color.
Seeds: Smooth; width 1 mm; length 2-3 mm; elliptic shape; brown in color.
Fruit: Explosive capsule; green in color.
   Anthers.—RHS 158A yellow white group.
   Pollen.—White in color.
   Stigma.—Nearly white RHS 157 D.
   Ovary.—Light green RHS 143 B.
Disease/Pest resistance/susceptibility: Unknown.

1. A new and distinct New Guinea Impatiens plant named ‘Danhardalil’, as illustrated and described herein.
